(19) United States
(12) Patent Application Publication
(10) Pub. No.: US 2019/0256392 A1
(43) Pub. Date: Aug. 22, 2019

(54) NEGATIVE HYDROGEN WATER FABRICATION DEVICE FOR THERAPY OF CANCEL, DIABETES, HYPERTENSION, CHOLESTEROL, TRIGLYCERIDE, PARKINSON'S DISEASE, AND ALZHEIMER'S DISEASE

(71) Applicant: Kuan-Chia Chen, New Taipei City (TW)

(72) Inventor: Kuan-Chia Chen, New Taipei City (TW)

(21) Appl. No.: 15/901,908

(22) Filed: Feb. 22, 2018

(51) Int. Cl. C02F 9/00; A61K 33/00

(52) U.S. Cl. CPC C02F 9/00; C02F 1/283; A61K 33/00

(57) ABSTRACT

A negative hydrogen water fabrication device includes a front-mounted filter assembly and a negative hydrogen ion water-activation device. The front-mounted filter assembly includes three resin filter cores that purify and supplies raw water to the negative hydrogen ion water-activation device in which eight activation filter cores, two nanometer gold/platinum filtered water generators, and an ultra-finning resonator are arranged for dissolving minerals in quantum form and trace elements that are needed for human bodies into water for generating, through application of unique physical characteristics, negative hydrogen potential alkaline water that is also subjected to oscillation dissociation to reduce water molecule clusters thereby providing active energy water having high penetration power and dissolution power for drinking by a user for activating cells of human body, enhancing metabolism and improving body quality.

### (a) TECHNICAL FIELD OF THE INVENTION

The present invention relates generally to a negative hydrogen water fabrication device, which provides a function of enhancing purity of water, generates alkaline water having negative hydrogen potential, and enables water to store energy for activating cellular functions to thereby offering and serving as antioxidant.

### (b) DESCRIPTION OF THE PRIOR ART

Water is one of the most constituent substances of biological bodies and is also important living resources for all creatures. Water takes around 60-70 percent of a human body an a human body must take around 2-3 liters of water everyday in order to maintain metabolism of the human body and regulate physiological functions and internal chemical reactions. Thus, water resources are vital for maintaining lives.

Due to the increasingly severe pollution or contamination of water, when a person take the polluted or contaminated water into the body, heavy metals, chemical toxicants, agrochemicals, radioactive particles, and other harmful substances will accumulate inside the human body and affect human health. It is commonly known that chloride has been used for disinfection of tap water and would generates, after tap water so disinfected is boiled, trihalomethanes that is considered hepatotoxicity, nephrotoxicity, and carcinogenicity and long inhalation of an excessive amount would cause severe damage to metabolism of the human body and may result in bladder cancers and colorectal cancers or even miscarriages of pregnant women. In view of such reasons, a number of households use water purification devices to have water filtered by filters arranged in the water purification devices for the purposes of acquiring pure water for drinking

On the other hands, it has been known, from a huge number of biochemical papers and reports, negative hydrogen ions that include an additional electron in the atom thereof and are negative ions carrying negative electricity provide an superbly strong power of oxidation resistance, and, when applied to a human body, would offer a function of regulating balance between positive and negative ions inside the human body to effectively clear off active oxygen radicals, which are primitive causes of various diseases, enhance activity of cells, improve natural healing power, and keep cells healthy.

The conventional household water purification devices, however, provide no effect of activating negative hydrogen in the water supplied therefrom and thus are incapable of providing or achieving the above described effects of health caring.

### SUMMARY OF THE INVENTION

The present invention provides a negative hydrogen water fabrication device, which purifies water and provides minerals and trace elements that are necessary for human bodies so as to provide an effect of preventing chronic diseases of human bodies, such as neuropathic pains, gout, diabetes, hypertension, hyperlipidemia, stone diseases, and cardiovascular diseases, and also to increase life kinetic energy by improving human body functions and activating cells, postpones aging, and improving symptoms, such as constipation and insomnia.

To achieve the above objective, the negative hydrogen water fabrication device of the present invention comprises a front-mounted filter assembly and a negative hydrogen ion water-activation device. The front-mounted filter assembly has a water ingress port connected to a pipe of raw water supply and a water egress port connected to a water introduction pipe of the negative hydrogen ion water-activation device and comprises three resin filter cores for removing impurities from water and absorbing harmful substances, such as chemicals and heavy metal contaminants, to purify water that is then supplied to the negative hydrogen ion water-activation device. The negative hydrogen ion water-activation device comprises eight activation filter cores, nanometer gold/platinum filtered water generators, an ultra-finning resonator, and a flow meter and is connected, through a discharge pipe, to a faucet. When water is supplied trough each of the activation filter cores and the nanometer gold/platinum filtered water generators, minerals in quantum form and trace elements may dissolve in water to generate negative hydrogen potential alkaline water through application of unique physical properties, and water molecule cluster can be made smaller through micro pressure oscillation principle for activation to thereby provide active energy water having high power of penetration and dissolution for drinking by a user.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a front view of the present invention.

FIG. 4 is another perspective view of the present invention.

FIG. 5 is a rear view of the present invention.

FIG. 6 is a cross-sectional view showing a nano-gold filtered water generator of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

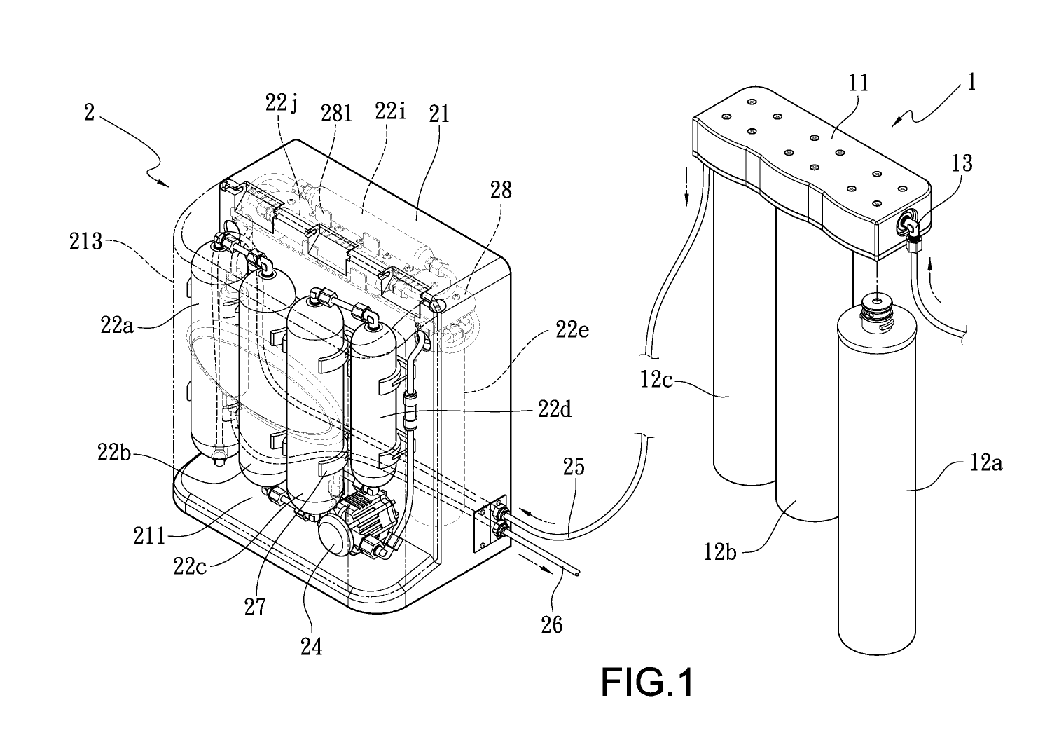

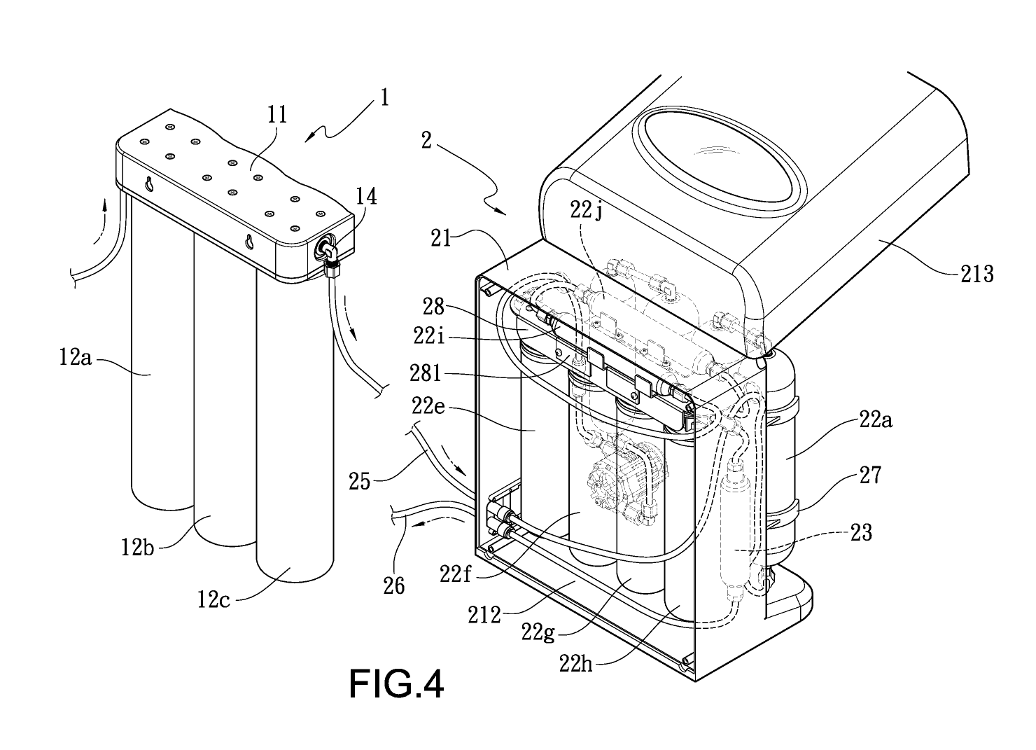

Referring to FIGS. 1, 2, and 4, the present invention comprises a front-mounted filter assembly 1 and a negative hydrogen ion water-activation device 2. The front-mounted filter assembly 1 comprises a base 11. The base 11 comprises three resin filter cores 12a-12c mounted thereon and is provided, on two sides thereof, with a water ingress port 13 and a water egress port 14 respectively. The water ingress port 13 is connected, through pipes, to a raw water supply source. The water egress port 14 is connected to a water introduction pipe 25 of the negative hydrogen ion water-activation device 2. The negative hydrogen ion water-activation device 2 comprises a housing 21 having an interior space in which eight activation filter cores 22a-22h that are formed of different materials and arranged in cascade, two nanometer gold/platinum filtered water generators 22i, 22j, an ultra-filming resonator 23, and a flow meter 24 are mounted. Water from the water supply source is first filtered by the front-mounted filter assembly 1 and then flows into the negative hydrogen ion water-activation device 2 to pass through the plurality of activation filter cores 22a-22h and the ultra-finning resonator 23 to generate negative hydrogen ion activated water to be supplied through a discharge pipe 26 of the negative hydrogen ion water-activation device 2 to a faucet for drinking

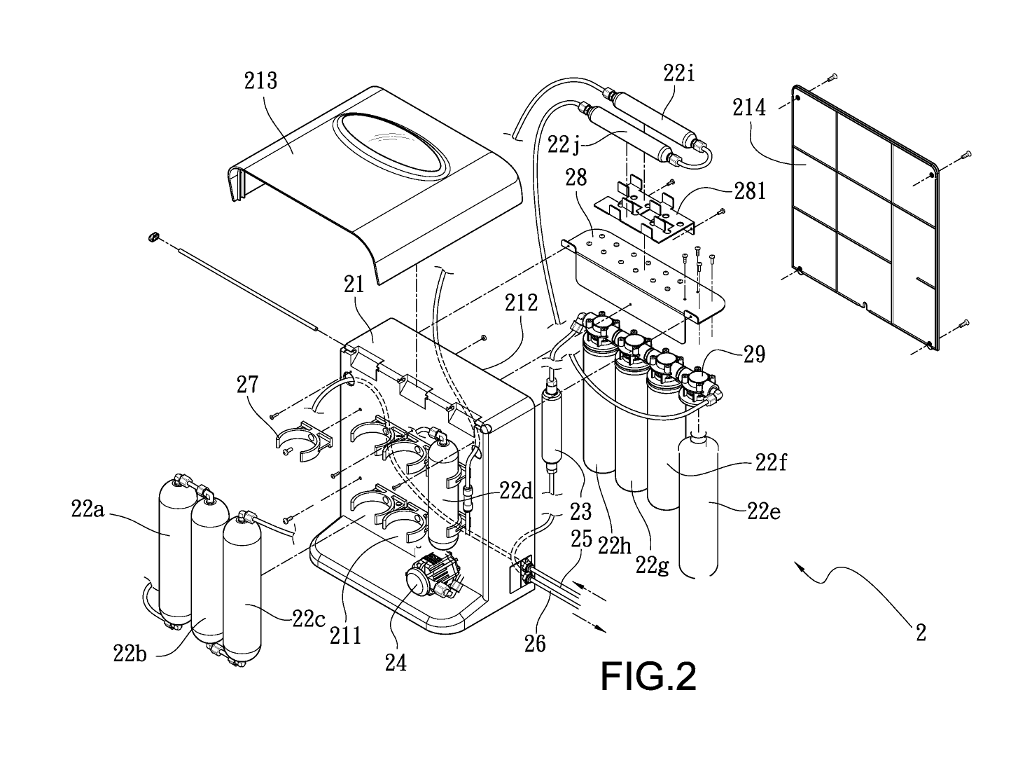

Referring to FIG. 2, the negative hydrogen ion water-activation device 2 is provided with a first receiving space 211 and a second receiving space 212 respectively formed in a front side and a rear side of the housing 21. The first receiving space 211 comprises a spring clip 27 for retaining and securing four activation filter cores 22a-22d, such that a leading terminal of the filter cores is connected to the water introduction pipe 25 and a tail terminal is connected to the flow meter 24, and the first receiving space is covered by a liftable front cover 213. The second receiving space 212 of the rear side is provided with a top board 28 having a top to which a retention support 281 is mounted for carrying and mounting the nanometer gold/platinum filtered water generators 22i, 22j that are in communication with each other. The top board 28 has a bottom to which coupling seats 29 that are arranged and connected in cascade are mounted for fast securing and fixing the other four filter cores 22e-22h with pipes arranged to connect one nanometer gold/platinum filtered water generator 22i to the flow meter 24. Another one nanometer gold/platinum filtered water generator 22j is connected to one terminal of the coupling seats 29, while another terminal of the coupling seats 29 is connected through ultra-finning resonator 23 to the discharge pipe 26. The second receiving space of rear side is covered by a rear cover 214.

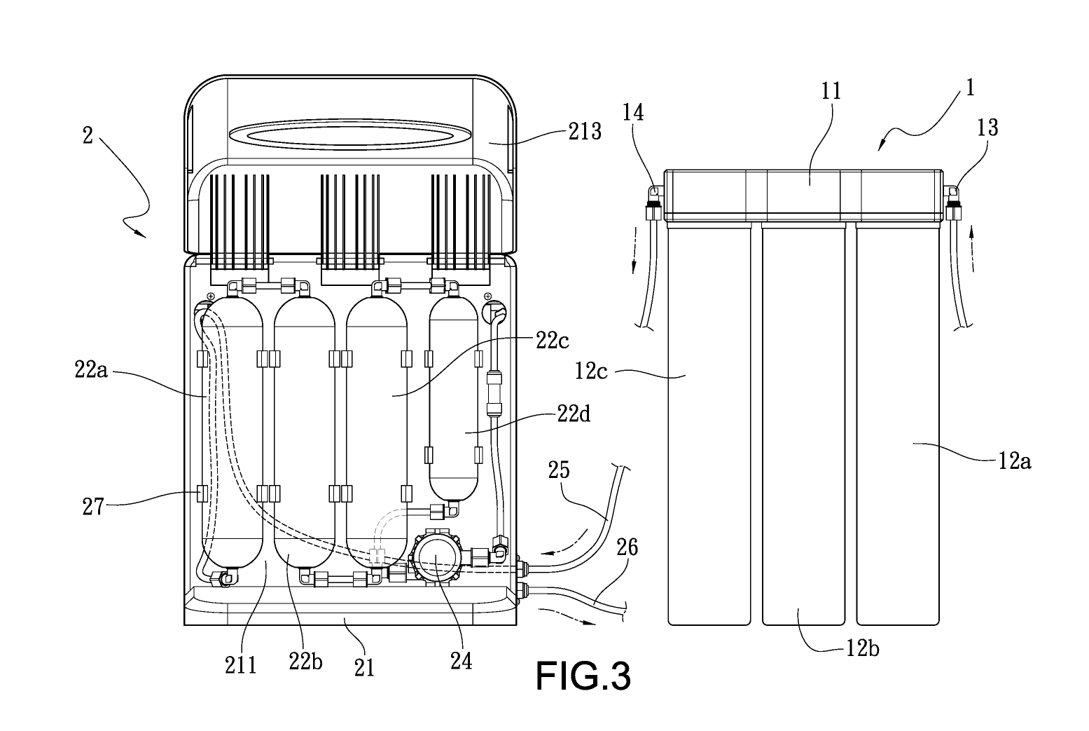

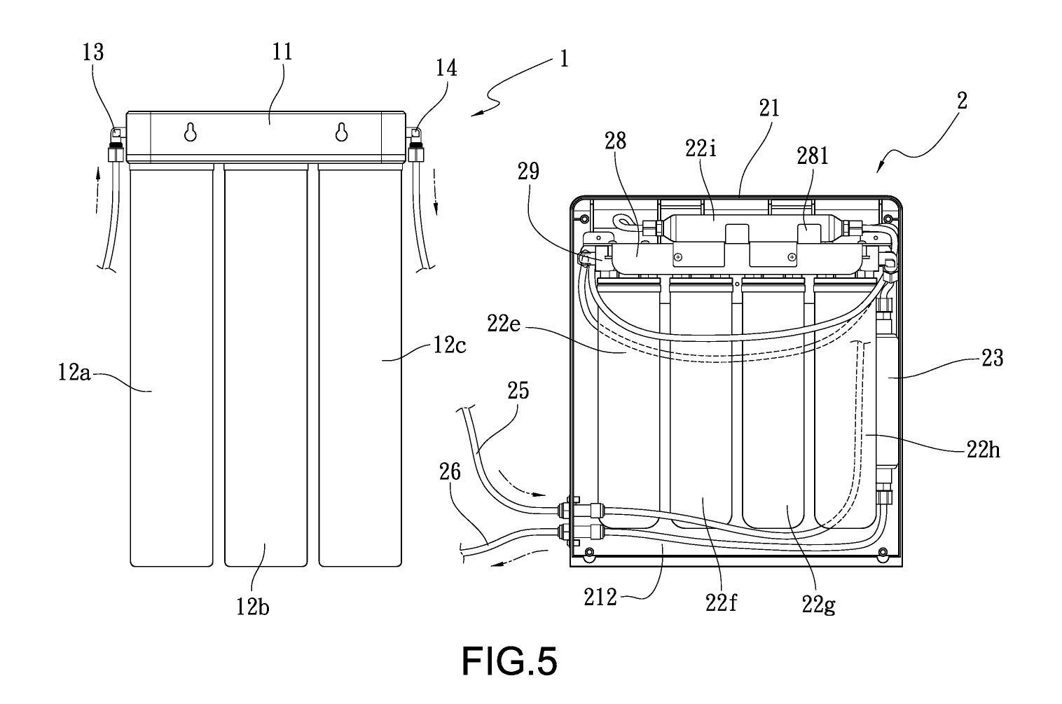

Referring to FIGS. 1 and 3-5, the three front-mounted resin filter cores 12a, 12b, 12c of the front-mounted filter assembly 1 and the eight activation filter cores 22a, 22b, 22c, 22d and 22e, 22f, 22g, 22h of the negative hydrogen ion water-activation device 2 each have a different function.

Among the front-mounted the resin filter cores, the first filter core 12a comprises an ultra-density fibrous polypropylene (PP) cotton filtration core using filtration fibers having pore size of 1 micron to provide an effect of high performance and low pressure difference for removing particle impurities that are visible to bare eyes, such as dust, earth, hair, iron rust, organic substance, algae, and floating microorganisms for the purpose of primary filtration of impurities contained in water to reduce the loading of the activation filter cores.

The second filter core 12b comprises a food grade ion filtration core that removes heavy metals that are harmful to human bodies, such as cadmium, and carcinogenic toxicants, such as arsenic, chlorides, and organic substances, and also to remove calcareous substances from water, lower hardness of water, and make water clear and smooth.

The third filter core 12c comprises an active carbon high-performance filtration core, which absorbs hazardous chemical substances, such as chlorine, odor, impurities, agrochemicals, and heavy metal contaminants, to achieve the purposes of purifying water.

The first activation filter core activation filter core 22a of the negative hydrogen ion water-activation device 2 comprises a trace element filtration core, wherein the trace elements are multiple minerals and trace elements comprising calcium, magnesium, sodium, and potassium that are released from energy stones formed by burning, at a high temperature, rare mountain minerals and may enable alkaline activity of water that passes therethrough to provide an effect of enhancing blood circulation and improving immunity.

The second activation filter core 22b comprises a negative hydrogen potential generator, which comprises nanometer hydrophilic catalytic material to generate, through application of unique physic properties, negative hydrogen ion energy water having a PH value of weak alkaline, demonstrating the capability of oxidation and reduction to effectively change a positive potential (around +300 orp) of the raw water (running water supplied through utility piping), through a predetermined mechanism, into water of negative potential (−300 orp), wherein water molecule cluster is split, through dissociation, into small molecule cluster, with enhanced dissolving power and penetration power to help activation of cell, enhance absorption of nutrition and metabolism of wastes, and neutralize excessive acids and free radicals to balance acidic/alkaline quality of human body and enhance physiological functions.

The three activation filter core 22c comprises a high oxygen content generator, which uses natural tourmaline composite elements to make energy mineral ceramics (burnt at 1350 degrees) to release ultrahigh oxygen content vibration energy, generating water of small molecules cluster having high oxygen content and energy and generating, through a sinistral quantum resonance mechanism, water-soluble calcium chloride after decomposition thereby providing an effect of decomposing calcium oxalate.

The fourth activation filter core 22d comprises π-water magnetic resonator, which uses magnetic waves to make water molecule clusters resonating for re-activation and prevention of water scale and to reduce water molecule clusters for making π-water and also carrying energy for activating water, increasing dissolved oxygen and dissolving power, activating cell for metabolism of body waste and toxicants, enhancing blood circulation, and improving supply of nutrition and enzyme to provide effects of improving metabolism, enhancing immunity, and improving body quality, and also showing antibacterial effect, to thereby provide natural healthy good-quality water.

The fifth activation filter core 22e comprises a malfan stone filtration core. Malfan stones are stones of volcano eruption and are natural stone-like minerals formed through tens of thousands of years, containing various trace elements that are beneficial for human body, such as calcium, magnesium, and sodium, and potassium, and may effectively absorb harmful heavy metals contained in water, such as chromium, copper, cadmium, and arisen, and residual chlorine contained in water, so that water, after passing therethrough, can be mineralized to release multiple minerals and trace elements necessary to human bodies and regulate pH value of water, and increase oxygen content to form alkaline active ionized water to provide great help to human bodies.

The sixth activation filter core 22f comprises sinistral π-water filtration core, which comprises far infrared minerals that are made nanometer size and then re-sintered to offer compression energy and functions to oscillate, divide, and activate water through application of ultra-intense energy for generating water of small molecule clusters having high dissolved oxygen and energy to quickly bring nutrition, high oxygen and energy into cells to thereby activate cells and regulate metabolism of human bodies.

The seventh activation filter core 22g comprises a KDF active carbon filtration core, wherein KDF designates a high purity copper alloy filtration material that makes use of oxidation and reduction of electrochemistry to remove contaminants of water and is a filter made by mixing chips of copper and zinc for effectively removing heavy metals and acidic ions to improve activation of water, providing effects of lowering bacterium growth speed and removing chlorine, wherein active carbon has high capability of absorbing free chlorine, disinfectant, and chemical residues contained in water and may remove dichloroethylene, bleaching agents, agrochemicals, trihalomethane, odors, chlorine, magnesium, organic substance, and other harmful chemicals and makes water weak alkaline.

The eighth activation filter core 22h comprises a nano-silver bacterium-suppressing filtration core, wherein activity of silver ions is improved through high-tech nanometer technology for combination with active carbon to improve the power of absorption of active carbon for organic polymeric substances by several time to effectively suppress and eliminate bleeding of bacteria in water and absorb harmful chemical substances to thereby ensure that water may achieve a standard for direct drinking

In addition to the eight activation filter cores 22a-22h, a nanometer ultra-finning resonator 23 is used in combination so that when drinking water that has been purified through the above-discussed filtration cores passes though a filtration core of the nanometer ultra-filming resonator 23, aeration of water can be generated through micro pressure oscillation principle to reduce and activate the water molecule clusters for drinking by a user, making it easy absorbed by human bodies to achieve the effects of activation of human body cells and improving metabolism. Further, the negative hydrogen ion water-activation device 2 of the present invention further comprises a flow meter 24 that provides a function of precaution alarm. With the arrangement of the flow meter 24, the total amount of filtered water can be measured so that when consumable materials, such as individual filtration cores and energy elements, reach a state of saturation, the supply of water is cut off to remind the user of replacement of these consumable materials, in order to ensure safety of drinking water.

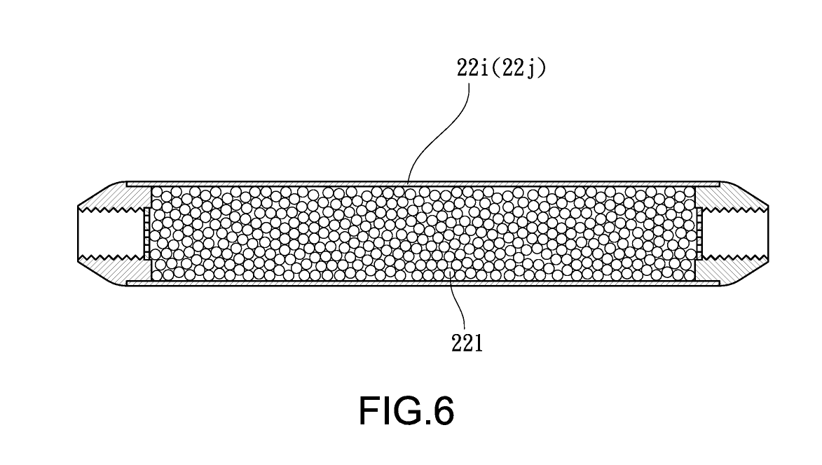

Referring to FIGS. 2 and 4-6, the negative hydrogen ion water-activation device 2 of the present invention comprises nanometer gold/platinum filtered water generators 22i, 22j, which are each filled therein with composite ceramic energy filter material (as shown in FIG. 6). The ceramic energy filter material 221 of one nanometer gold/platinum filtered water generator 22i contain nanometer gold particles that are formed through application of nanometer technology to gold. The other nanometer gold/platinum filtered water generator 22j comprises nanometer platinum particles that are formed by making platinum nanometer in size. The gold and platinum nanometer particles are added in a clay base material and sintered to form a composite ceramic energy filter material 221 containing gold and platinum nanometer particles. The fabrication of the composite ceramic energy filter material 221 can be carried out in such a way that basic materials of natural minerals, such as far infrared powder or malfan stone powder or tourmaline powder or argutite powder, can be added in the clay material for sintering and forming.

The two nanometer gold/platinum filtered water generators 22i, 22j provide not only an effect of purification of water, but also an effect of removal of impurity and harmful substances, such as chemicals and heavy metals, from water, and also help suppress growth of bacteria, activate water, and release minor amount of nanometer gold and platinum to provide the capability of oxidation resistance to water, reduction of molecule clusters, and supply of pure energy water having high penetration power and oxidization resistance for drinking by a user. The nanometer gold energy water, when entering a human body, provides a function of fast activating cells and fast absorption of minerals and nutrition necessary to human body and discharge unnecessary wastes and toxicants out of human body to thereby achieve effects of regulating physiological functions, preventing aging, and improving health, and also helping improve body quality, activating cells, improving immunity, reducing the likelihood of hyperlipidemia, hypertension, and hyperglycemia, gout, chronic diseases of nerve system, liver, and kidneys, and cancels.

In summary, the present invention supplies pure and contaminant-free water that has proper hardness and good penetration and absorption and good quality and also helps activates water to provide minerals and trace elements that are needed for human bodies, and may regulate body quality, enhance physiological function to allow a user to drink water with safety and health, thereby providing practical utilization.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

1. A negative hydrogen water fabrication device for generating negative hydrogen water, which carries an additional electrode in an atom thereof to form negative ions that help improve resistance against oxidation in human body and are applicable to improve cancel, diabetes, hypertension, cholesterol, triglyceride, Parkinson's disease, and Alzheimer's disease, the negative hydrogen water fabrication device comprising a front-mounted filter assembly and a negative hydrogen ion water-activation device,
wherein the front-mounted filter assembly comprises, at least, three resin filter cores for filtering water and is provided with a water ingress portion adapted to connect to a raw water supply pipe to receive raw water from the raw water supply pipe and a water egress portion for supplying filtered water on two sides thereof respectively; and
the negative hydrogen ion water-activation device comprises a housing, the housing being provided, at suitable locations thereon, with a water introduction pipe that is connected to the water egress port of the front-mounted filter assembly to receive the filtered water from the front-mounted filter assembly and a discharge pipe adapted to connect to a faucet, wherein eight activation filter cores comprising different filtration materials, two nanometer gold/platinum filtered water generators, an ultra-fine cluster generation water-resonator, and a flow meter are connected, in cascade between the pipes, wherein the filtered water that is formed by feeding the raw water through the front-mounted filter assembly and supplied to the water introduced pipe of the negative hydrogen ion water-activation device is allowed to flow through the activation filter cores and the water resonator for conversion into negative hydrogen water that is dispensable through the discharge pipe and the faucet.

2. The negative hydrogen water fabrication device according to claim 1, wherein the housing of the negative hydrogen ion water-activation device comprises a first receiving space and a second receiving space respectively formed in front and rear sides thereof, wherein the first receiving space comprises a spring clip to retain four of the eight activation filter cores, which have a leading terminal connected to the water introduction pipe and a tail terminal connected to the flow meter, and is covered by a liftable front cover; and the second receiving space comprises a top board having a top to which the two nanometer gold/platinum filtered water generators that are in communication with each other are mounted, the top board having a bottom to which a coupling seat is mounted for retaining the other four of the eight activation filter cores, wherein one of the nanometer gold/platinum filtered water generators is connected, through a pipe, to the flow meter and another one of the nanometer gold/platinum filtered water generators is connected to a terminal of the coupling seat, the coupling seat having another terminal that is connected, through the ultra-fine cluster generation water-resonator, to the discharge pipe, a rear cover being set to cover the second receiving space.
